(19) United States
(12) Patent Application Publication
(10) Pub. No.: US 2009/0118756 A1
(43) Pub. Date: May 7, 2009

(54) INTRA-GASTRIC BALLOON PROVIDED WITH A GEL-CONTAINING VALVE, KIT AND USE THEREOF

(76) Inventors: Nicolas Francois Michel Valencon, Pont Eveque (FR); Pascal Paganon, Serezin du Rhone (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/916,181

(22) PCT Filed: Jun. 1, 2005
(86) PCT No.: PCT/FR2005/001345

§ 371 Date: Jul. 25, 2008

(51) Int. Cl. A61M 29/02

(52) U.S. Cl. 606/192

(57) ABSTRACT

The invention relates to an intra-gastric balloon designed to be implanted inside the stomach of a patient for the purpose of reducing the volume of the stomach by way of treatment for obesity, said intra-gastric balloon (1) comprising:
at least one bag (2) suitable for being filled, at least partially, with an inflation fluid;
connection means (3), mounted on said bag (2) and suitable for receiving a connection member (11) for connection to an inflation fluid source;
said intra-gastric balloon being characterized in that the connection means (3) include a chamber (16) designed to be transpierced by said connection member (11) with a view to injecting the inflation fluid into said bag (2), said chamber (16) containing a sealing fluid that is sufficiently viscous and/or thick to guarantee that the connection means (3) are leaktight once the connection member (11) has been removed.

The invention also provides an intra-gastric balloon for treating obesity.

### TECHNICAL FIELD

The present invention relates to the general technical field of devices that are implantable in the human body and that are designed to be used in treating obesity, in particular morbid obesity, and the present invention relates especially to implants that are suitable for artificially reducing the volume of the stomach, in particular with a view to procuring a feeling of satiety in the patient.

The present invention relates more particularly to an intra-gastric balloon designed to be implanted inside the stomach of a patient for the purpose of reducing the volume of the stomach by way of treatment for obesity, said intra-gastric balloon comprising:

at least one bag suitable for being filled, at least partially, with an inflation fluid; and

connection means, mounted on said bag and suitable for receiving a connection member for connection to an inflation fluid source.

The present invention also relates to a kit for deploying an intra-gastric balloon designed to be implanted in the stomach of a patient by way of treatment for obesity, said kit comprising:

an intra-gastric balloon; and

a connection member for connecting the intra-gastric balloon to an inflation fluid source.

The present invention also relates to novel use of a viscous fluid in connection means for connecting an intra-gastric balloon to a fluid source.

### PRIOR ART

In order to treat patients suffering from obesity, it is known that intra-gastric balloons can be used that are designed to be implanted in the stomachs of the patients in order to reduce the space available for food, with a view, in particular to procuring a feeling of satiety.

Such a known intra-gastric balloon generally comprises at least one flexible bag suitable for taking up a collapsed (or small-volume) position enabling the intra-gastric balloon to be implanted via the oral route.

The bag is designed to be filled, once the intra-gastric balloon is implanted in the stomach, with an inflation fluid, e.g. with air or with physiological fluid, in a manner such as to impart to the intra-gastric balloon its operational shape, in which it occupies a volume inside the stomach that is sufficient to occupy a large portion of the space available for food.

The bag is generally made of materials having good impermeability properties so as to prevent the fluid contained in the bag from progressively leaking into the stomach and so as to prevent the intra-gastric balloon from deflating. In particular, the leaktightness of the bag must be sufficient for the intra-gastric balloon to maintain its operational shape throughout the period of the treatment that can vary from a few weeks to several months.

In known intra-gastric balloons, the bag is provided with connection means, such as a valve, designed to enable the intra-gastric balloon to be connected to an inflation fluid source, via a connection member comprising, for example, a catheter and an inflation needle. The connection means further comprise means for guaranteeing that it is leaktight, once the connection member has been withdrawn, in order to prevent the fluid contained in the bag from leaking out.

When the inflation fluid is formed by a liquid, such as a physiological fluid, the means used can be in the form of a “duck-bill” valve. A duck-bill valve is designed in such a manner as to allow pressurized fluid to flow from the outside towards the inside of the intra-gastric balloon, and to prevent fluid from flowing in the opposite direction. In general, such valves make it possible to obtain good results with liquids, but they are generally unsatisfactory when the inflation fluid is formed by a gas. The leaktightness procured with that type of valve is then no longer satisfactory. Valves of the “septum” type are then preferred. Such a septum valve is formed by a membrane that is made of silicone, that can be of various thicknesses, that is suitable for being transpierced by an inflation needle, and that has self-healing characteristics.

Intra-gastric balloons equipped with valves of the “septum” type are simple to manufacture and are generally satisfactory, but they do however suffer from various non-negligible drawbacks.

Firstly, in the event of prolonged storage, an aging phenomenon can appear in the silicone forming the septum. In particular, the septum can become dry and brittle, and lose its elasticity and its impermeability properties under the effect of such aging.

In addition, intra-gastric balloons are sometimes sold in kits, such a kit including an inflation needle pre-installed on the balloon, the inflation needle being pre-positioned, in its operational position, so as to pass through the septum and so as to open out into the bag of the intra-gastric balloon.

Such kits can be stored in sterile or non-sterile housings for prolonged periods before being used. Unfortunately, the prolonged presence of the inflation needle through the septum can give rise to formation of an orifice which, if the storage period is very long, can remain after the needle has been removed, once the intra-gastric balloon has been inflated. Such an orifice then constitutes a preferred passage via which fluid contained in the intra-gastric balloon can leak out, thereby causing the balloon to deflate.

### SUMMARY OF THE INVENTION

Objects assigned to the invention are thus to remedy the various above-listed drawbacks, and to propose a novel intra-gastric balloon of leaktightness that is greater than the leaktightness of prior art intra-gastric balloons, even after a prolonged period of storage.

Another object of the invention is to propose a novel intra-gastric balloon that is suitable for being connected easily, in leaktight manner, and entirely safely to an inflation fluid source.

Another object of the invention is to propose a novel intra-gastric balloon that is particularly lightweight and well tolerated by the patient.

Another object of the invention is to propose a novel intra-gastric balloon that can be extracted easily and rapidly via the oral route.

Another object of the invention is to propose a novel intra-gastric balloon that is suitable for being grasped easily and securely by endoscopic extractor tools.

Objects assigned to the invention are also to propose a novel kit comprising an intra-gastric balloon and a connection member for connecting the balloon to an inflation fluid source, and that, even after a long period of storage, offers good leaktightness properties.

Objects assigned to the invention are also to propose novel use of a viscous fluid making it possible to improve the leaktightness of an intra-gastric balloon.

The objects assigned to the invention are achieved by means of an intra-gastric balloon designed to be implanted inside the stomach of a patient for the purpose of reducing the volume of the stomach by way of treatment for obesity, said intra-gastric balloon comprising:

at least one bag suitable for being filled, at least partially, with an inflation fluid; and

connection means, mounted on said bag and suitable for receiving a connection member for connection to an inflation fluid source;

said intra-gastric balloon being characterized in that the connection means include a chamber designed to be transpierced by said connection member with a view to injecting the inflation fluid into said bag, said chamber containing a sealing fluid that is sufficiently viscous and/or thick to guarantee that the connection means are leaktight once the connection member has been removed.

The objects assigned to the invention are also achieved by means of a kit for deploying an intra-gastric balloon designed to be implanted in the stomach of a patient by way of treatment for obesity, said kit comprising:

an intra-gastric balloon of the invention, and in particular, as described above; and

a connection member for connecting the intra-gastric balloon to the inflation fluid source, which connection member is, in its storage configuration, mounted in a manner such as to transpierce said chamber so as to open out into the bag.

The objects assigned to the invention are also achieved by means of the use of a viscous and/or thick fluid as a seal in connection means mounted on an intra-gastric balloon and designed to enable the intra-gastric balloon to be connected in leaktight manner to an inflation fluid source.

### BRIEF DESCRIPTION OF THE DRAWING

Other objects and advantages of the invention will appear on reading the following description, and on examining the accompanying drawing which is given merely by way of non-limiting illustration and in which:

FIG. 1 is a section view showing an intra-gastric balloon of the invention that has a single bag;

FIG. 2 is a section view showing a variant embodiment of an intra-gastric balloon of the invention that has two concentric bags;

FIG. 3 is a perspective view showing connection means of the invention that are designed to be mounted on an intra-gastric balloon; and

FIG. 4 is a section view on line A-A of FIG. 3, showing the connection means shown in FIG. 3.

### BEST MANNER OF IMPLEMENTING THE INVENTION

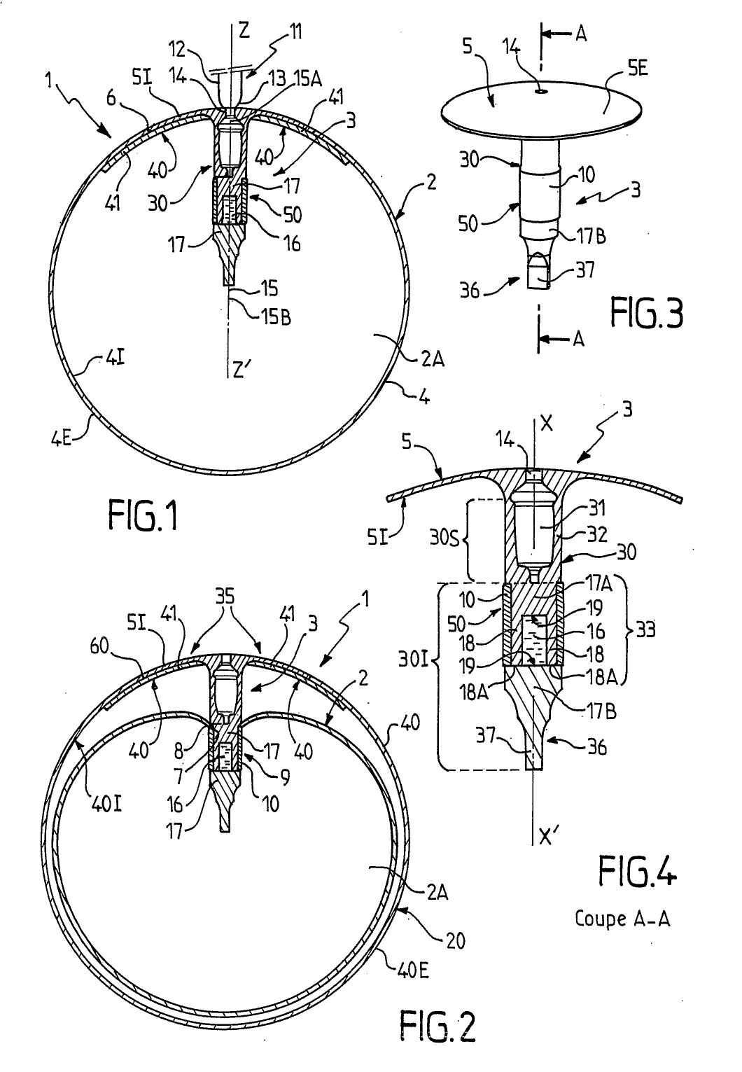

FIGS. 1 and 2 show two variant embodiments of an intra-gastric balloon 1 of the invention in its operational configuration, i.e. in its expanded configuration, in which it occupies a large portion of the space available for food in the stomach.

The intra-gastric balloon 1 of the invention advantageously has the characteristic of being expandable, i.e. it is made of flexible materials, and preferably of elastomer materials such as silicone or elastomer thermoplastic polyurethane, enabling it to expand from a collapsed configuration (not shown), in which it occupies a small volume enabling it to be implanted via the oral route, to an expanded configuration in which it occupies an operational volume (FIGS. 1 and 2).

The intra-gastric balloon 1 of the invention comprises at least one bag 2 suitable for being filled, at least partially, with an inflation fluid, and defining an internal volume 2A.

The intra-gastric balloon 1 of the invention further comprises at least one set of connection means 3 mounted on said bag 2 and designed to be connected to a fluid source (not shown) by means of a connection member 11 in order to cause the intra-gastric balloon 1 to expand inside the stomach by being filled with the inflation fluid.

In a first embodiment of the invention shown in FIG. 1, the intra-gastric balloon 1 comprises a single bag 2 forming a surface casing 4 for the intra-gastric balloon 1. In preferred manner, the bag 2 is made of a bio-compatible flexible material, and preferably of medical-grade silicone.

In this first embodiment, the connection means 3 are mounted on the bag 2 and are preferably secured thereto, e.g. by bonding with adhesive or by heat-sealing. To this end, the connection means 3 are provided with a collar 5 designed to enable the connection means 3 to be fastened to the bag 2 in leaktight manner, e.g. by bonding the collar 5 with adhesive or by heat-sealing it to the periphery of a hole 6 provided through the bag 2.

In a second embodiment shown in FIG. 2, the intra-gastric balloon 1 of the invention comprises at least first and second bags 2, 20 that are preferably flexible, the first bag 2 being disposed inside the second bag 20 so as to form an inner first bag 2 and an outer second bag 20. In this variant, the connection means 3 are fastened in leaktight manner to the first bag 2, and are more precisely mounted in a passage 7 defined by a neck 8 extending towards the inside of the first bag 2. More precisely, the connection means 3 are fastened to the first bag 2 by means of a fastening element 9 that is preferably formed by a ring 10. The fastening element 9 is thus advantageously shaped and disposed so as to exert pressure on the neck 8 that is sufficient to pinch it between the connection element 3 and the fastening element 9.

In the second embodiment of the invention, the first bag 2 forms means for deploying the second bag 20, said second bag then forming the surface casing 40 of the intra-gastric balloon 1. The inflation fluid, and in particular air, is advantageously fed into the first bag 2, thereby causing it to inflate. In the manner of a tire inner tube, the first bag 2 expanding in turn causes the second bag 20 forming the surface casing 40 of the intra-gastric balloon 1 to expand and to be deployed.

Advantageously, the first and second bags 2, 20 are made of materials that are mutually different and that are not necessarily compatible.

The expression “non-compatible” refers to materials that are particularly difficult or even impossible to assemble together conventionally by adhesive bonding or by heat-sealing, in view of industrial production constraints and of medical requirements as regards quality and safety.

Preferably, the first and second bags 2, 20 are made of elastomer materials, the first bag 2 preferably being made of a material having a gas barrier effect, such as elastomer thermoplastic polyurethane, and the second bag 20 preferably being made of a biocompatible material having good strength, such as silicone. The use of a barrier-effect material for the first bag 2 advantageously makes it possible to reduce the thickness of the first bag 2 while preserving or even improving the leaktightness of the balloon.

In the second embodiment of the invention, the periphery of the collar 5 is preferably bonded with adhesive or heat-sealed to the hole 60 provided through the second bag 20, the connection means 3 thus closing off in leaktight manner the second bag 20, i.e. the surface casing 40 of the intra-gastric balloon 1. The two-bag configuration shown in FIG. 2 is preferred to the one-bag configuration shown in FIG. 1 because it makes it possible to obtain an intra-gastric balloon 1 that has strength and leaktightness that are higher than those of single-bag balloons, while remaining compact in the collapsed state, in particular by means of the small thickness of the inner bag.

The connection member 11 advantageously comprises a catheter 12 serving to provide fluid connection between the intra-gastric balloon 1 and the inflation fluid source and designed to be connected to the intra-gastric balloon 1 by means of an end-piece 13 inserted into a recess 14 provided in the connection means 3. The connection member 11 further comprises an inflation needle 15 that is preferably formed by a hollow needle having one of its ends 15A secured to the end-piece 13 and its other end 15B, which is preferably atraumatic, situated inside the internal volume 2A. As shown in FIG. 1, the inflation needle is inserted through the connection means 3 along a perforation axis Z-Z′.

In accordance with the invention, the connection means 3 include a chamber 16 designed and advantageously disposed to be transpierced by the connection member 11, and more precisely by the inflation needle 15 with a view to injecting inflation fluid into the bag 2, said chamber 16 containing a sealing fluid that is sufficiently viscous and/or thick to make the connection means 3 leaktight once the connection member 11 has been removed. Thus, after the connection member 11, and in particular the inflation needle 15 has been withdrawn, the sealing fluid contained in the chamber 16 is displaced and comes to occupy the space left by the inflation needle 15. The chamber 16, which is advantageously fully filled with sealing fluid, then constitutes a self-closing member suitable for closing automatically in leaktight manner after the inflation needle 15 has been removed.

The expression “sealing fluid” is used herein to designate any type of fluid or semi-solid substance that is sufficiently viscous and/or thick to perform a sealing function. It can be a liquid, characterized by its viscosity, or indeed a semi-solid, characterized by its thickness and its resistance to penetration, measured by means of a penetrometer. Thus, for certain fluids (or semi-solids) having high thickness or high viscosity, such as creams or gels, for which measurements using a standard viscometer are difficult and unreliable, use is often made of penetration measurements that consist in measuring, at 25° C., the depth to which a moving body of determined weight and size penetrates over a fixed time.

The connection means 3 advantageously include a main body 30 that is preferably substantially elongate and that projects into the intra-gastric balloon 1, and in particular into the internal volume 2A. The chamber 16 is then preferably provided inside the main body 30.

Advantageously, the connection means 3, and in particular the main body 30, have at least one self-healing partition 17 disposed in axial alignment with the chamber 16, in a manner such as to be transpierced by the connection member 11, said self-healing partition 17 being sufficiently flexible and elastic to self-close once the connection member 11 has been removed (FIGS. 1 and 2).

The expression “self-healing partition” is used to designate a wall or a membrane made of a material that is sufficiently flexible to be transpierced by the connection member 11, and in particular by the inflation needle 15, and that has elasticity and shape memory properties that are sufficient to close the orifice left after the inflation needle 15 has been removed, thereby forming a leaktight partition.

Particularly advantageously, and as shown in FIGS. 1 and 2, the connection means 3 include at least two self-healing partitions 17 disposed at either end of the chamber 16, in axial alignment therewith.

The main body 30 preferably extends longitudinally towards the inside of the internal volume 2A, along a longitudinal axis X-X′, substantially coinciding with the perforation axis Z-Z′. When the intra-gastric balloon 1 is positioned in the vertical configuration shown in FIG. 1, the longitudinal axis X-X′ substantially coincides with the vertical direction, the connection means 3 then being situated in the top portion of the intra-gastric balloon 1.

Advantageously, and as shown in FIG. 4, the connection means 3 include a top self-healing partition 17A and a bottom self-healing partition 17B. The terms “bottom” and a “top” refer to the relative positioning of the self-healing partitions 17 when the intra-gastric balloon 1 is in the vertical configuration shown in FIG. 1. The self-healing partitions 17 are preferably made of an elastomer material such as solid and flexible silicone of medical grade. The top self-healing partition 17A and the bottom self-healing partition 17B can advantageously be made of materials that are identical or that are different, and, for example, of silicones of different hardnesses. Preferably, the entire connection means 3 are made of silicone.

The chamber 16 is advantageously defined axially, i.e. along the longitudinal axis X-X′ along which the main body 30 extends, by the top and bottom self-healing partitions 17A and 17B. The chamber 16 is also defined laterally, i.e. in a plane that is substantially perpendicular to the longitudinal axis X-X′ by one or more side walls 18.

Preferably, the side walls 18 have the characteristic of being leaktight and are preferably formed by one or more self-healing membranes. The chamber 16 is then isolated in leaktight manner from the internal volume 2A and relative to the outside of the balloon, by leaktight walls serving to prevent fluid communication between firstly the chamber 16 and the internal volume 2A and secondly the outside of the intra-gastric balloon.

Advantageously, the chamber 16 has puncture walls 19 preferably formed by self-healing partitions 17, and in particular by the top and bottom self-healing partitions 17A and 17B. The expression “puncture walls” is used herein to designate the walls of the chamber 16 at which the inflation needle 15 transpierces the chamber 16.

Naturally, it is possible to imagine implementing a chamber 16 whose walls, and in particular whose puncture walls 19 are not formed by self-healing walls. In which case, the passage provided through the puncture walls 19 of the chamber 16 once the connection member 11 has been removed must be of sufficiently small diameter to prevent the sealing fluid contained in the chamber 16 from leaking out. An inflation needle 15 that is particularly fine and a sealing fluid that is sufficiently viscous to remain confined in the chamber 16 are then chosen.

Advantageously, the sealing fluid has viscosity and/or thickness sufficient to trap the inflation fluid particles, e.g. gas bubbles or drops of physiological fluid, that might form in the chamber 16 once the connection member 11 has been removed. Thus, on being removed, the connection member 11, and in particular the inflation needle 15, vacates a passage through the puncture walls 19. The resulting passage can, if it is not automatically closed off, constitute a leakage orifice for the inflation fluid contained in the internal volume 2A. This phenomenon can, in particular be observed when the puncture walls 19 of the chamber 16 are not formed by self-healing partitions 17.

However, this phenomenon can also be observed when the chamber 16 is defined by self-healing partitions 17, in particular if the inflation needle 15 remains through the self-healing partitions 17 for a long time. In which case, degradation might be observed in the self-healing characteristic of the self-healing partitions 17.

The sealing fluid contained in the chamber 16 then plays an essential part because it makes it possible to trap any inflation fluid particles that might escape from the chamber 16 through the above-mentioned passage.

Preferably, the viscosity and the thickness of the sealing fluid are such that, at 25° C., the depth to which a moving body of 1 inch and of 12 grams (g) penetrates into the sealing fluid with a fall time of 15 seconds is less than 20 millimeters (mm), and preferably lies in the range 10 mm to 18 mm.

Penetration is measured by a penetrometer of the Lab Line I0005 type or of an equivalent type, at 25° C.

Even more preferably, the sealing fluid is formed by a gel, e.g. by a silicone gel of the NuSil type (reference MED 12-6300), that comprises two components and that cross-links (cures) after being put in place in the chamber 16. The cross-linking of the gel is preferably performed at a temperature of 140° C., for about 5 hours. The chamber 16 is advantageously filled in a plurality of steps, in order to make up for the shrinkage progressively, each filling step being followed by a cross-linking step.

Advantageously, the main body 30 comprises a top portion 30S that is hollow so as to form a cavity 31, e.g. a cylindrical cavity, and a bottom portion 30I inside which the chamber 16 is formed.

It is interesting to note that the presence of the cavity 31 in the connection means 3 is independent of whether or not the chamber 16 is present. The purpose of the cavity 31 is merely to facilitate taking hold of the intra-gastric balloon 1 when explanting it, and said cavity could thus be associated with any type of self-closing member and, for example, with a single self-healing partition, such as the top self-healing partition 17A.

In order to facilitate taking hold of the intra-gastric balloon 1, the top portion 30S is thus hollow over a distance sufficient for the wall (or the walls) of the main body 30 defining the cavity 31 to be able to fold over while the top portion 30S of the main body 30 is being pinched with an extractor tool (not shown) for extracting the intra-gastric balloon 1, such as endoscopic forceps.

Preferably, the cavity 31 extends longitudinally, i.e. along the longitudinal axis X-X′, over a length greater than its mean width (or than its diameter, when the cavity is a cylindrical cavity) measured in a direction that is substantially perpendicular to the longitudinal axis X-X′.

By way of illustrative and non-limiting example, the cavity 31 can be of length greater than 1.5 times its mean width.

The length of the cavity 31 advantageously results from a compromise between:

firstly the need to limit the total length of the connection means 3 and of the main body 30 so as not to hinder implantation of the intra-gastric balloon 1 via the oral route; and

secondly, the need to be able to pinch the connection means 3 in the vicinity of the surface casing 4, 40 of the intra-gastric balloon 1 in order to enable said balloon to be extracted.

Thus, the intra-gastric balloon 1 is implanted while the inflation needle 15 is mounted in the connection means 3 in a manner such as to pass longitudinally through the main body 30. The main body 30 and the inflation needle 15 thus form a rigid assembly having very low longitudinal flexibility. However, in order to insert the intra-gastric balloon 1 into the oral passageways of the patient, the surgeon requires a certain amount of flexibility, hence the need to minimize the length of the main body 30.

Particularly advantageously, the collar 5 extends radially to the surface of the intra-gastric balloon 1 in a manner such as to form a graspable zone 35 via which the intra-gastric balloon 1 can be taken hold of by the extractor tool (not shown).

It is possible to imagine making the collar 5 of a material having hardness greater than the hardness of the material forming the surface casing 4, 40, e.g. of hard silicone, so as to offer, at least locally, higher strength, and so as to prevent it from being transpierced by the extractor tool. However, it is preferable, in order to enable the intra-gastric balloon 1 to collapse, to make the collar 5 of a flexible material of hardness equivalent to or only very slightly greater than the hardness of the surface casing 4, 40.

Preferably, the graspable zone 35 is associated with a reinforcement 41 (FIGS. 1 and 2) for forming a reinforced graspable zone 35 dedicated to extracting the intra-gastric balloon 1. The reinforcement 41 is advantageously in the form of a flexible membrane 42 that extends along the graspable zone 35 in a manner such that it does not project towards the outside of the surface casing 4, 40, and thus does not form any protuberance that might be detrimental to the uniform and atraumatic geometrical shape of the intra-gastric balloon 1.

Preferably, the shape of the reinforcement 41 substantially matches the shape of the collar 5 and the shape of the surface casing 4, 40, the reinforcement being associated with said collar and with said casing. Even more preferably, the reinforcement 41 is superposed on the collar 5 inside the intra-gastric balloon 1 and is advantageously bonded with adhesive or heat-sealed over its entire surface to the inside surface 51 of the collar 5. The reinforcement 41 can also be extended, as shown in FIGS. 1 and 2, under the surface casing 4, 40. In which case, the reinforcement 41 is preferably secured to the surface casing 4, 40 by being heat-sealed or bonded with adhesive to the inside face 4I, 40I of said surface casing 4, 40. It is also possible, without going beyond the ambit of the invention, to imagine mounting the reinforcement 41 on the outside surface 5E of the collar or indeed embedding the reinforcement 41 in the thickness of the collar 5. Similarly, the reinforcement 41 may extend over the outside face 4E, 40E of the surface casing 4, 40 or be embedded in the thickness thereof.

The reinforcement 41 advantageously comprises at least one piece of textile, comprising, for example, a polyester net, or indeed a woven (or non-woven) fabric made from polyamide fibers and/or aramid fibers. It is also possible to imagine implementing, as a reinforcement piece, a fibrous structure of the “honeycomb” type that is well known per se.

Advantageously, the connection means 3 include a compression member 50, such as a ring, disposed in a manner such as to surround at least one of the self-healing partitions 17 in a manner such as to compress it laterally. The compression member 50 is advantageously dimensioned to exert inward radial compression on said self-healing partition 17, in a direction that is substantially perpendicular to the perforation axis Z-Z′, in a manner such as to impart thereto its leaktight and self-healing characteristics, or merely such as to contribute to improving the self-healing characteristic intrinsic to said self-healing partition 17.

Preferably, the compression member 50 and the fastening member 9 are formed by the same element, namely the ring 10.

Advantageously, the connection means 3, and more precisely the main body 30 are in the form of a cylindrical elongate body that includes a compressed segment 33 including at least one self-healing partition 17, preferably formed by the top self-healing partition 17A, and the chamber 16 defined laterally by the side walls 18. The compressed segment 33 is advantageously compressed laterally and inwardly by means of the compression member 50 formed by the ring 10.

Preferably, the compression of the chamber 16 lies in the range 10% to 20%.

The bottom self-healing partition 17B is advantageously mounted on the compressed segment 33, and more precisely on the ends 18A of the side walls 18, in a manner such as to close the chamber 16 in leaktight manner. In the preferred variant shown in FIG. 4, the bottom self-healing wall 17B is not compressed radially by the compression member 50 and finds itself in a relaxed state.

Preferably, the side walls 18 of the chamber 16 are formed integrally with one of the self-healing partitions, and preferably with the top self-healing partition 17A, forming therewith a one-piece unit suitable for being obtained by molding.

Even more preferably, the collar 5, the top portion 30S, and the compressed segment 33 form a one-piece unit obtained by molding, the bottom self-healing partition 17B being separate and being mounted by adhesive bonding or by heat-sealing on the compressed segment 33 so as to close the chamber 16.

Advantageously, the connection means 3, and in particular the main body 30, include a pull member 36 suitable for being manipulated for axially pulling the connection means 3 along the longitudinal axis X-X′, and for enabling the compression means 50 to be mounted around at least one of the self-healing partitions 17.

Thus, the compression member 50 advantageously has an inside diameter smaller than the outside diameter at rest of the self-healing partition 17 with which it is associated so that it can radially compress said partition.

Axially pulling the self-healing partition 17 with which the compression member 50 is associated along the longitudinal axis X-X′ thus makes it possible to reduce the outside diameter of the self-healing partition 17 until said outside diameter reaches the inside diameter of the compression member 50 and in particular of the ring 10. It is thus possible to fit the ring 10 over the self-healing partition, and in particular over the top self-healing partition 17A, as shown in FIG. 4.

Preferably, the pull element 36 is formed by a traction tab 37 that is secured to or integral with the connection means 3, and more particularly to or with the bottom self-healing partition 17B (FIG. 4). The traction tab 37 thus advantageously forms an extension of the main body 30 along the longitudinal axis X-X′.

The invention also relates to a kit for deploying an intra-gastric balloon 1 that is designed to be implanted in the stomach of a patient by way of treatment for obesity.

In accordance with the invention, the kit comprises an intra-gastric balloon 1 of the invention, and a connection member 11 for connecting the intra-gastric balloon 1 to an inflation fluid source, which connection member, in its storage configuration, is mounted in a manner such as to transpierce the chamber 16 in order to open out in the bag 2. The connection member 11 advantageously comprises an inflation needle 15 that is preferably formed by a hollow needle suitable for transpiercing said chamber 16. Throughout the period of storage, the inflation needle 15 is mounted in a manner such as to pass through the chamber 16 and through any self-healing partitions 17 disposed in alignment therewith.

The present invention also relates to novel use of a viscous and/or thick fluid as a seal inside the connection means 3 mounted on an intra-gastric balloon 1 and designed to enable the intra-gastric balloon 1 to be connected in leaktight manner to an inflation fluid source.

Preferably, the viscosity and the thickness of the sealing fluid are such that, at 25° C., the depth to which a moving body of 1 inch and of 12 g penetrates into the sealing fluid with a fall time of 15 seconds is less than 20 mm, and preferably lies in the range 10 mm to 18 mm.

Even more preferably, the sealing fluid is formed by a gel, such as a silicone gel of the NuSil type whose reference is, for example, MED 12-6300.

The design of the intra-gastric balloon 1 of the invention thus enables the intra-gastric balloon 1 inside the stomach of the patient to be filled safely, with no risk of leakage, even after a prolonged period of storage of the intra-gastric balloon 1 or of the kit comprising, in association, the intra-gastric balloon 1 and the connection member 11 mounted on the balloon.

Another advantage of the intra-gastric balloon 1 of the invention results from its ease of extraction, by means of the presence of the cavity 31 whose length is sufficient to enable the connection means 3 to be pinched by endoscopic forceps.

### SUSCEPTIBILITY OF INDUSTRIAL APPLICATION

The invention is susceptible of industrial application in manufacturing and using intra-gastric balloons for treating obesity.

1. An intra-gastric balloon designed to be implanted inside the stomach of a patient for the purpose of reducing the volume of the stomach by way of treatment for obesity, said intra-gastric balloon (1) comprising:
at least one bag (2) suitable for being filled, at least partially, with an inflation fluid; and
connection means (3), mounted on said bag (2) and suitable for receiving a connection member (11) for connection to an inflation fluid source;
said intra-gastric balloon being characterized in that the connection means (3) include a chamber (16) designed to be transpierced by said connection member (11) with a view to injecting the inflation fluid into said bag (2), said chamber (16) containing a sealing fluid that is sufficiently viscous and/or thick to guarantee that the connection means (3) are leaktight once the connection member (11) has been removed.

2. An intra-gastric balloon according to claim 1, characterized in that the sealing fluid is of viscosity and/or of thickness sufficient to trap inflation fluid particles, e.g. bubbles or drops, that might form in the chamber (16) once the connection member (11) has been removed.

3. An intra-gastric balloon according to claim 1, characterized in that the viscosity and the thickness of the sealing fluid are such that, at 25° C., the depth to which a moving body of 1 inch and of 12 g penetrates into said sealing fluid with a fall time of 15 seconds is less than 20 mm, and preferably lies in the range 10 mm to 18 mm.

4. An intra-gastric balloon according to claim 1, characterized in that the sealing fluid is formed by a gel, e.g. a silicone gel.

5. An intra-gastric balloon according to claim 1, characterized in that the connection means (3) include at least one self-healing partition (17) disposed in axial alignment with the chamber (16) in a manner such as to be transpierced by the connection member (11), said self-healing partition (17) being sufficiently flexible and elastic to self-close, once the connection member (11) has been removed.

6. An intra-gastric balloon according to claim 5, characterized in that the connection means (3) include at least two self-healing partitions (17) disposed at either end of the chamber (16) and in axial alignment therewith.

7. An intra-gastric balloon according to claim 5, characterized in that the chamber (16) includes puncture walls (19), and in that the puncture walls (19) are formed by self-healing partitions (17).

8. An intra-gastric balloon according to claim 5, characterized in that the connection means (3) include a compression member (50), of the ring type, disposed in a manner such as to surround at least one of the self-healing partitions (17) in a manner such as to compress it laterally.

9. An intra-gastric balloon according to claim 8, characterized in that the connection means (3) include a pull member (36) suitable for being manipulated for axially pulling the connection means (3) and for enabling the compression member (50) to be mounted around at least one of the self-healing partitions (17).

10. An intra-gastric balloon according to claim 9, characterized in that the pull member (36) is formed by a traction tab (37) fastened to or formed integrally with the connection means (3).

11. An intra-gastric balloon according to claim 1, characterized in that the connection means (3) include a main body (30) projecting into the intra-gastric balloon (1), said main body (30) including a top portion (30S) that is hollow so as to form a cavity (31), and a bottom portion (301), inside which the chamber (16) is provided.

12. An intra-gastric balloon according to claim 11, characterized in that the top portion (30S) is hollow over a distance sufficient for the wall (32) of the main body (30) defining the cavity (31) to fold over while the top portion (30S) of the main body (30) is being pinched with a tool for extracting the intra-gastric balloon (1), such as endoscopic forceps.

13. An intra-gastric balloon according to claim 1, characterized in that the connection means (3) are provided with a collar (5) extending radially at the surface of the intra-gastric balloon (1) so as to form a graspable zone (35) via which the intra-gastric balloon (1) can be taken hold of by the extractor tool.

14. An intra-gastric balloon according to claim 13, characterized in that the graspable zone (35) is associated with a reinforcement (41) so as to form a reinforced graspable zone (35) dedicated to extracting the intra-gastric balloon (1).

15. A kit for deploying an intra-gastric balloon (1) designed to be implanted in the stomach of a patient by way of treatment for obesity, said kit comprising:
an intra-gastric balloon according to claim 1; and
a connection member (11) for connecting the intra-gastric balloon (1) to the inflation fluid source, which connection member is, in its storage configuration, mounted in a manner such as to transpierce said chamber (16) so as to open out into the bag (2).

16. A kit according to claim 15, characterized in that the connection member (11) includes an inflation needle (15) suitable for transpiercing said chamber (16).

17. The use of a viscous and/or thick fluid as a seal in connection means (3) mounted on an intra-gastric balloon (1) and designed to enable the intra-gastric balloon (1) to be connected in leaktight manner to an inflation fluid source.

18. A use according to claim 17, characterized in that the viscous and/or thick fluid is formed by a gel, e.g. a silicone gel.
